SEQUENCE LISTING (33 sequences)

SEQ ID NO: 1 - protein, 7 aa, Artificial sequence
  synthetic
SNYLYDN

SEQ ID NO: 2 - protein, 9 aa, Artificial sequence
  synthetic
REFPFYGDY

SEQ ID NO: 3 - protein, 6 aa, Artificial sequence
  synthetic
EKHHSH

SEQ ID NO: 4 - protein, 38 aa, Artificial sequence
  synthetic
DSHEKRHHGYRRKFHEKHHSHREFPFYGDYGSNYLYDN

SEQ ID NO: 5 - protein, 27 aa, Artificial sequence
  synthetic
RKFHEKHHSHREFPFYGDYGSNYLYDN

SEQ ID NO: 6 - protein, 32 aa, Artificial sequence
  synthetic
DSHAKRHHGYKRKFHEKHHSHRGYRSNYLYDN

SEQ ID NO: 7 - protein, 15 aa, Artificial sequence
  synthetic
EKHHSHREFPFYGDY

SEQ ID NO: 8 - protein, 17 aa, Artificial sequence
  synthetic
REFPFYGDYGSNYLYDN

SEQ ID NO: 9 - protein, 19 aa, Artificial sequence
  synthetic
SHREFPFYGDYGSNYLYDN

SEQ ID NO: 10 - protein, 21 aa, Artificial sequence
  synthetic
HHSHREFPFYGDYGSNYLYDN

SEQ ID NO: 11 - protein, 23 aa, Artificial sequence
  synthetic
EKHHSHREFPFYGDYGSNYLYDN

SEQ ID NO: 12 - protein, 25 aa, Artificial sequence
  synthetic
FHEKHHSHREFPFYGDYGSNYLYDN

SEQ ID NO: 13 - protein, 19 aa, Artificial sequence
  synthetic
RKFHEKHHSHREFPFYGDY

SEQ ID NO: 14 - protein, 15 aa, Artificial sequence
  synthetic
EKHHSHREFPFYGDY

SEQ ID NO: 15 - protein, 21 aa, Artificial sequence
  synthetic
RKFHEKHHSHREFPFYGDYGS

SEQ ID NO: 16 - protein, 17 aa, Artificial sequence
  synthetic
EKHHSHREFPFYGDYGS

SEQ ID NO: 17 - protein, 23 aa, Artificial sequence
  synthetic
RKFHEKHHSHREFPFYGDYGSNY

SEQ ID NO: 18 - protein, 19 aa, Artificial sequence
  synthetic
EKHHSHREFPFYGDYGSNY

SEQ ID NO: 19 - protein, 25 aa, Artificial sequence
  synthetic
RKFHEKHHSHREFPFYGDYGSNYLY

SEQ ID NO: 20 - protein, 21 aa, Artificial sequence
  synthetic
EKHHSHREFPFYGDYGSNYLY

SEQ ID NO: 21 - protein, 17 aa, Artificial sequence
  synthetic
RKFHEKHHSHREFPFYG

SEQ ID NO: 22 - protein, 13 aa, Artificial sequence
  synthetic
EKHHSHREFPFYG

SEQ ID NO: 23 - protein, 19 aa, Artificial sequence
  synthetic
HHSHREFPFYGDYGSNYLY

SEQ ID NO: 24 - protein, 17 aa, Artificial sequence
  synthetic
SHREFPFYGDYGSNYLY

SEQ ID NO: 25 - protein, 15 aa, Artificial sequence
  synthetic
SHREFPFYGDYGSNY

SEQ ID NO: 26 - protein, 15 aa, Artificial sequence
  synthetic
FPFYGDYGSNYLYDN

SEQ ID NO: 27 - protein, 13 aa, Artificial sequence
  synthetic
FYGDYGSNYLYDN

SEQ ID NO: 28 - protein, 15 aa, Artificial sequence
  synthetic
HHSHREFPFYGDYGS

SEQ ID NO: 29 - protein, 13 aa, Artificial sequence
  synthetic
SHREFPFYGDYGS

SEQ ID NO: 30 - protein, 24 aa, Artificial sequence
  synthetic
DSHAKRHHGYKRKFHEKHHSHRGY

SEQ ID NO: 31 - protein, 14 aa, Artificial sequence
  synthetic
GYKRKFHEKHHSHR

SEQ ID NO: 32 - protein, 11 aa, Artificial sequence
  synthetic
SHREFPFYGDY

SEQ ID NO: 33 - protein, 44 aa, Artificial sequence
  synthetic
GGDSHEKRHHGYRRKFHEKHHSHREFPFYGDYGSNYLYDNLPET

(19) United States
(12) Patent Application Publication
(10) Pub. No.: US 2017/0239330 A1
(43) Pub. Date: Aug. 24, 2017

(54) FORMULATIONS FOR HISTATIN THERAPEUTICS

(71) Applicant: RAPID PATHOGEN SCREENING, INC., Sarasota, FL (US)

(72) Inventors: Robert P. Sambursky, Bradenton, FL (US); Robert W. Vandine, Montoursville, PA (US); Peter Condon, Longboat Key, FL (US); Uma Mahesh Babu, Bradenton, FL (US)

(73) Assignee: RAPID PATHOGEN SCREENING, INC., Sarasota, FL (US)

(21) Appl. No.: 15/519,217

(22) PCT Filed: Oct. 8, 2015
(86) PCT No.: PCT/US15/54585

§ 371 Date: Apr. 14, 2017

Related U.S. Application Data
(60) Provisional application No. 62/064,164, filed on Oct. 15, 2014.
(60) Provisional application No. 62/064,137, filed on Oct. 15, 2014.
(60) Provisional application No. 62/064,151, filed on Oct. 15, 2014.
(60) Provisional application No. 62/065,911, filed on Oct. 20, 2014.
(60) Provisional application No. 62/065,920, filed on Oct. 20, 2014.
(60) Provisional application No. 62/065,935, filed on Oct. 20, 2014.

(51) Int. Cl. A61K 38/17; A61K 31/20; A61F 9/008; A61K 31/573; A61K 31/65; A61K 38/13; A61K 31/56

(52) U.S. Cl. CPC A61K 38/1709; A61K 38/13; A61K 31/20; A61K 31/56; A61K 31/573; A61K 31/65; A61F 9/00836

(57) ABSTRACT

Histatin formulations include at least two different histatins. Histatins are used before or after laser surgery. Histatins are also used to treat ocular viruses, fungi or parasites. Histatins may be used for corneal wound healing and as a treatment for ocular surface disease in humans and other animals. Histatin formulations for treating ocular wounds or ocular surface disease include a first peptide comprising a first histatin and a second peptide comprising a second histatin. A method of performing corneal laser surgery on at least one eye of a patient includes cutting a cornea of the eye of the patient with a surgical laser and administering a therapeutic amount of at least one medicament comprising at least one peptide to an ocular surface of the eye.

### REFERENCE TO RELATED APPLICATIONS

This application claims one or more inventions which were disclosed in Provisional Application No. 62/064,164, filed Oct. 15, 2014, entitled “FORMULATIONS FOR HISTATIN PROTECTIVES AND THERAPEUTICS”, Provisional Application No. 62/064,137, filed Oct. 15, 2014, entitled “FORMULATIONS FOR HISTATIN THERAPEUTICS”, Provisional Application No. 62/064,151, filed Oct. 15, 2014, entitled “FORMULATIONS FOR HISTATIN THERAPEUTICS”, Provisional Application No. 62/065,911, filed Oct. 20, 2014, entitled “FORMULATIONS FOR HISTATIN THERAPEUTICS” and Provisional Application 62/065,920, filed Oct. 20, 2014, entitled “FORMULATIONS FOR HISTATIN THERAPEUTICS” and Provisional Application No. 62/065,935, filed Oct. 20, 2014, entitled “FORMULATIONS FOR HISTATIN PROTECTIVES AND THERAPEUTICS”. The benefit under 35 USC §119(e) of the United States provisional applications is hereby claimed, and the aforementioned applications are hereby incorporated herein by reference.

### BACKGROUND OF THE INVENTION

Field of the Invention

The invention pertains to the field of therapeutics. More particularly, the invention pertains to histatins as ocular therapeutics.

Description of Related Art

Histatins have been shown in in vitro studies to be wound healing agents from saliva. More specifically, WO 2009/087117 (and its US equivalent US Patent Publication 2011/0178010), herein incorporated by reference, identified peptides of histatin, which had wound healing properties in vitro.

Histatin 1 (Hst-1) and Histatin 2 (Hst-2) have been identified as major wound-closing factors in human saliva (“Discovery of the Wound Healing Capacity of Salivary Histatins”, thesis of Menno Johannes Oudhoff, Academic Centre for Dentistry Amsterdam (ACTA), VU University Amsterdam and University of Amsterdam, The Netherlands, 2010, herein incorporated by reference). These studies were all done in vitro and cannot be translated to a finding for therapeutic or clinical use, especially since wound and disease healing are complex processes that need to be highly regulated in order to function properly.

### SUMMARY OF THE INVENTION

Histatins may be used for corneal wound healing and as a treatment for ocular surface disease in humans and other animals. For example, histatins may be included in eye drops, eye gels, ointment, glue, or embedded in (polymer) contact lenses.

Histatin formulations for treating ocular wounds or ocular surface disease include a first peptide comprising a first histatin selected from the group consisting of histatin 1, a fragment of histatin 1, histatin 2, a fragment of histatin 2, histatin 1 and histatin 2, histatin 1 and a fragment of histatin 2, histatin 2 and a fragment of histatin 1, or a fragment of histatin 1 and a fragment of histatin 2, and a second peptide comprising a second histatin selected from the group consisting of histatin 5 and a fragment of histatin 5. In some embodiments, the formulation includes approximately 50-100 μg/mL of a combined formulation of the first histatin and the second histatin. In some embodiments, the formulation includes 50-75 wt % of the first histatin and 25-50 wt % of the second histatin with respect to a total weight of histatins in the formulation. In some embodiments, the weight-to-weight ratio of histatin 1 to histatin 5 is 1:1, 2:1, 3:1, 4:1, 5:1, 1:2, 1:3, 1:4, or 1:5.

A method of performing corneal laser surgery on at least one eye of a patient includes cutting a cornea of the eye of the patient with a surgical laser and administering a therapeutic amount of at least one medicament comprising at least one peptide to an ocular surface of the eye. The at least one peptide is selected from the group consisting of a peptide comprising at least a first histatin, a peptide comprising at least a fragment of the first histatin, a first peptide comprising at least the first histatin and a second peptide comprising at least the second histatin, a first peptide comprising at least the fragment of the first histatin and a second peptide comprising at least the fragment of the second histatin, a first peptide comprising at least the first histatin and a second peptide comprising at least the fragment of the second histatin, or a first peptide comprising at least the fragment of the first histatin and a second peptide comprising at least the second histatin. In some embodiments, the method further includes the step of performing a dry eye test on the eye of the patient prior to performing surgery. If the dry eye test gives a positive result, an anti-inflammatory formulation is preferably administered prior to performing surgery. The anti-inflammatory formulation preferably includes an anti-inflammatory agent selected from the group consisting of at least a first histatin, at least a fragment of the first histatin, at least the first histatin and a second histatin, at least the fragment of the first histatin and a fragment of the second histatin, at least the first histatin and at least the fragment of the second histatin, or at least the second histatin and at least the fragment of the first histatin.

A method of identifying whether a patient that is scheduled to undergo ocular surgery has dry eye on at least one eye of the patient includes the steps of performing a dry eye test on at least one eye of the patient and, if the dry eye test gives a positive result, administering a therapeutic amount of an anti-inflammatory formulation comprising at least one peptide comprising at least one histatin to an ocular surface of at least one eye prior to ocular surgery. The peptide is selected from the group consisting of a peptide comprising at least a first histatin, a peptide comprising at least a fragment of the first histatin, a first peptide comprising at least the first histatin and a second peptide comprising a second histatin, a first peptide comprising at least the fragment of the first histatin and a second peptide comprising at least the fragment of the second histatin, a first peptide comprising at least the first histatin and a second peptide comprising at least the fragment of the second histatin, or a first peptide comprising at least the fragment of the first histatin and a second peptide comprising at least the second histatin.

A method of treating a fungus, a virus, or a parasite on an ocular surface includes the step of administering a therapeutic amount of at least one medicament comprising a first peptide and second peptide to the ocular surface, wherein the first peptide comprises a first histatin or a fragment of the first histatin and the second peptide comprises a second histatin or a fragment of the second histatin.

### DETAILED DESCRIPTION OF THE INVENTION

Histatins are naturally occurring oral peptides produced by humans and non-human primates that demonstrate direct anti-infective activity, potent anti-inflammatory properties, and stimulate epithelial wound healing in several tissue and organ culture systems. A research facility has developed a technique to isolate this natural substance, making it a potential topical treatment for wounds.

In a preferred embodiment, a peptide including at least one amino acid sequence of at least eight amino acids adjacently present in Histatin 1, 2, 3, and/or 5 is used to treat a corneal wound or ocular surface disease. In other preferred embodiments, multiple histatin peptides or peptide fragments are used.

US Patent Publications 2013/0310326, and 2013/0310327, both published on Nov. 21, 2013, and entitled “HISTATIN FOR CORNEAL WOUND HEALING AND OCULAR SURFACE DISEASE”, and herein incorporated by reference, disclose histatins that may be used for corenal wound healing and as a treatment for ocular surface disease in humans and other animals.

Since histatins are protease inhibitors, they can overcome the deleterious effects of such proteases as the collagenases (MMPs) and other proteolytic enzymes which are produced as a result of inflammatory diseases or if found in elevated levels for unknown causes. In addition, as general protease inhibitors, histatins could be a component of artificial tears or soothing drops or gels that are used for comfort rather than medical treatment.

The histatins are preferably administered using eye drops, gels, ointments including histatin, tissue glue, or by incorporating histatin into a contact lens worn by a patient. In a preferred embodiment, the therapeutic amount of histatin accelerates wound or ocular surface disease healing compared to corneal wounds or ocular surface diseases not treated with histatin.

The ocular surface diseases may include, but are not limited to, inflammatory dry eye disease, corneal ulcerations and erosions, inflammatory and infectious keratitis and conjunctivitis, surgical interventions, and trauma.

In some preferred embodiments, the histatin concentration is between approximately 0.1 μg/mL and approximately 1000 mg/mL. In other preferred embodiments, the histatin concentration is between approximately 0.1 μg/mL and 100 μg/mL. In still other preferred embodiments, the histatin concentration is between approximately 0.1 μg/mL and 10 μg/mL. In some preferred embodiments, the histatin concentration is greater than or equal to approximately 1 μM.

The administering step may be repeated multiple times per day and/or for a plurality of days. In one preferred embodiment, this step is repeated at least one time a day for a plurality of days. In another preferred embodiment, the step is repeated chronically at least one time a day. In some preferred embodiments, the step is repeated up to hourly for a plurality of days. In another preferred embodiment, the step is repeated at least two times a day for a plurality of days. In yet another preferred embodiment, the step is repeated at least three times a day for a plurality of days, for example for seven days. In another preferred embodiment, the step is repeated four times a day for five days.

In one preferred embodiment, the histatin is a peptide including 8 to 44 amino acids. In some preferred embodiments, the peptide is a L-peptide. In other preferred embodiments, the peptide is a cyclic peptide.

In some preferred embodiments, the amino acid sequence of the histatin peptide is one or more of SEQ ID NOS: 1 through 33, or any combinations of these sequences. In alternative embodiments, one or more of the amino acid sequences have a substitution, deletion and/or insertion of up to 3 amino acids. In other alternative embodiments, one or more of the amino acid sequences have a substitution, a deletion and/or an insertion of two or less amino acids. In other alternative embodiments, one or more of the amino acid sequences have a substitution, a deletion, and/or an insertion in one amino acid.

The SEQ ID NO: 4 peptide is also known as Histatin 1 (Hst-1). Note that the first serine in this amino acid sequence may be a phosphoserine. The SEQ ID NO: 5 peptide is also known as Histatin 2 (Hst-2, also equivalent to amino acids 12-38 of Hst-1). The SEQ ID NO: 6 peptide is also known as Histatin 3 (Hst-3). The SEQ ID NO: 30 peptide is also known as Histatin 5 (Hst-5). Parts and fragments of each of these amino acid sequences may be used, alone or in combination, including but not limited to SEQ ID NOS: 1-3, 7-29 (for Histatin 1, Histatin 2 and Histatin 3) and SEQ ID NO: 32 (for Histatin 5) to facilitate wound closure in the embodiments described herein. While the L stereoisomer of the amino acids is preferred for the amino acid sequences described herein, D stereoisomers may alternatively be used. Alternatively, amino acid sequences that include these histatins and other amino acids, for example SEQ ID NO: 33, which is a sortase cyclized histatin (including all of Histatin 1), may be used in the embodiments described herein. Any histatin sequences could be cyclized and used in the embodiments described herein.

In preferred embodiments, using histatin amino acid sequences that promote ocular wound closure or healing of ocular surface disease (for example, a histatin amino acid sequence present in histatin 1 or histatin 2) in combination with a histatin amino acid sequence with antimicrobial properties (for example, a histatin amino acid sequence present in histatin 5), has synergistic healing effects. This strategy combines the direct effects that histatin 1 and histatin 2 have on wound closure with the indirect effects the antimicrobial properties of histatin 5 have on wound closure. More specifically, histatins 1 and 2 promote wound closure, while histatin 5 prevents microbial infection, thereby creating a better environment for wound healing.

In one preferred embodiment, methods of ocular treatment include the step of administering a therapeutic amount of a peptide or a peptide fragment of at least two histatins to an ocular surface. The histatins are preferably administered using eye drops, gels, ointments including histatin, tissue glue, or by incorporating histatin into a contact lens worn by a patient. The peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

Some preferred embodiments use amino acid sequences from Hst-1 and/or Hst-2 in combination with amino acid sequences from Hst-5 to treat ocular surfaces or specifically for treating ocular wounds. In these embodiments, one or more amino acid sequences from Hst-1 and/or Hst-2 are chosen, and one or more amino acid sequences from Hst-5 are chosen. In some embodiments, the full length Histatin 1 (SEQ ID NO: 4), full length Histatin 2 (SEQ ID NO: 5), and/or the full length Histatin 5 (SEQ ID NO: 30) could be used. In other embodiments, portions of Hst-1, Hst-2, and/or Hst-5 could be used. For example, SEQ ID NO: 29, which is equivalent to amino acids 20-32 of Histatin 1, may be a preferred amino acid sequence to use for wound closure in some embodiments. In other examples, peptides including SEQ ID NO: 32, a peptide fragment of Histatin 1 and Histatin 2 that appears to be a core motif for wound closure, may be used. Other preferred sequences from Hst-1 and Hst-2 include, but are not limited to, SEQ ID NO: 8, SEQ ID NO: 9 and SEQ ID NO: 13. As another example, SEQ ID NO: 31, a fragment of Histatin 5 (Gusman et al., “Salivary Histatin 5 is an inhibitor of Both Host and Bacterial Enzymes Implicated in Periodontal Disease”, Infect. Immun 2001, 69(3): 1402, pp. 1402-1408, herein incorporated by reference), may be used, preferably in combination with Histatin 1 or Histatin 2 or fragments thereof. In other preferred embodiments, fragments of Hst-1 or Hst-2 are used with full length Hst-5 (SEQ ID NO: 30) or full length Hst-1 (SEQ ID NO: 4) or Hst-2 (SEQ ID NO: 5) are used with fragments of Hst-5 (for example, SEQ ID NO: 31). In yet other embodiments, any combination of fragments of Hst-1 and/or Hst-2, full length Hst-1 and/or Hst-2, fragments of Hst-5, or full length Hst-5 may be used. In some preferred embodiments, the concentration of the Hst-5 peptide used is greater than or equal to approximately 1 μM.

The amino acids and the peptides described herein may include at least one functional grouping (for example, an amine and/or carboxylic group) protected with a protective grouping in some embodiments. Since the peptides are applied to tissue, skin or a wound, a protected form of the peptide may be preferred to resist degradation. The form of protection needs to be biologically compatible and compatible with pharmaceutical use. Some examples include, but are not limited to, the acylation or the acetylation of the amino-terminal ends, cyclization or the amidation or the esterfication of the carboxy-terminal ends. Thus, the peptides described herein may be used in a protected form.

The peptides described herein may be made by traditional chemical synthesis, enzymatic synthesis, or any other method known in the art.

The peptides preferably include at least 8 amino acids. In one preferred embodiment, the peptides include a range of 8 to 44 amino acids, but the peptides may alternatively include more than 44 amino acids.

“Wounds”, as defined herein, are injuries to living tissue, and can be caused by a cut, blow, or other impact. In most wounds, the skin or another external surface is cut or broken. Healing of those wounds using histatins 1 and 2 occurs by cell migration (epithelial migration, closing of an epithelial defect) and/or tissue regeneration. The regeneration occurs without induction of mitosis. In some embodiments, a wound is more specifically a physical manifestation of a breakdown of the protective function of the skin or other outer part of the body that normally provides a protective function, such as, for example, the cornea. In some embodiments, the wound reflects a loss of continuity of the epithelium. A cause of a wound may include, but is not limited to, surgery, a blow, a cut, contact with one or more chemicals, heat, cold, friction, a shear force, pressure, an ulcer, or a carcinoma.

The histatins and the histatin sequences described herein may be used as ocular anti-fungal agents, ocular antiviral agents, anti-protozoan agents and/or ocular antiparasitic agents. In one embodiment, the histatins are used to treat herpetic eye diseases (such as herpes simplex keratitis caused by herpes simplex virus and herpes zoster ophthalmicus caused by the varicella-zoster virus). In another embodiment, the histatins are used to treat adenovirus infections. In yet another embodiment, the histatins may be used to treat protozoan acanthamoeba infection such as Acanthamoeba keratitis. Acanthamoeba keratitis is a rare, parasitic infection that threatens the patient's vision, and is most often seen in contact lens wearers. Acanthamoeba are one of the most common protozoa in soil, and can also be found in fresh water or other habitats. As a general protease inhibitor, histatins should also inhibit the functions of proteolytic toxin causing organisms, including, but not limited to, Anthrax and Salmonella.

The antiviral efficacy of a histatin may be verified through the following type of in vitro test: 150 μL of a solution containing one or more histatins and a control solution are aliquoted into separate sterile screw cap microfuge tubes. 150 μL of stock virus in phosphate-buffered saline (PBS) are added to each tube containing the compounds and are mixed. The drug containing and control tubes are then incubated at 37° C. At 60 minutes of incubation, 300 μL of fresh tissue culture medium containing 20% fetal bovine serum is added to the tubes. Standard viral plaque assays are immediately performed to determine the residual viral titers present in each sample. Viral titers (PFU+1) are Log₁₀ converted and Log₁₀ reductions in titers from the control are calculated for each trial. The mean±SD Log reduction in titer for each virus are calculated for the two trials. Mean reductions in titer of at least one Log₁₀ are considered effective reductions. Mean reductions in titer of three Log₁₀ (99.9%) are considered virucidal reductions.

The antiparasitic efficacy of a histatin may be verified through the following type of in vitro test: 0.1 mL of acanthamoeba inoculum is pipetted into 0.5 mL each of polyhexamethylene biguanide (PHMB) 0.02%, saline and two different concentrations of at least one histatin. The inoculated histatin and control samples are incubated at 30° C. for 24 hours. At 24 hours, 0.05 mL of the inoculated samples is removed from the mixtures and plated on non-nutrient agar overlaid with Enterobacter aerogenes using a glass rod to disperse the samples. This prevents a concentrated amount of histatin to inhibit bacterial growth. The overlay is prepared by spreading of 0.3 mL of the Enterobacter aerogenes slurry on a non-nutrient agar with a soft-tipped applicator. The plates are incubated at 30° C. in an air incubator. After another 24 hours, a second overlay of Enterobacter aerogenes is administered to assure the food source is available to the acanthamoeba without any effect of residual drug. All plates are monitored for the robust growth of acanthamoeba resulting in a mixture of sparse trophozoites and predominant cysts at days 7 and 14. Robust growth at day 7 terminates testing with a positive result. At day 14, all remaining plates (plates observed for the lack of robust growth) are vigorously sub-cultured with a soft-tipped applicator onto fresh non-nutrient agar overlaid with Enterobacter aerogenes. After 7 days incubation, all plates are monitored for the robust growth of acanthamoeba resulting in a mixture of sparse trophozoites and predominant cysts. For each drug concentration, positive growth is graded as a “1”, and no growth is denoted as “0”. Any incidence of positive growth after 24 hours is considered as survival. Negative growth at 24 hours is denoted as “kill”.

### In Vivo Studies

Efficacy studies of histatin for corneal wound healing utilize an animal model, namely rabbits. Histatins are naturally produced substances that stimulate healing in several tissue and organ culture systems. The results of these studies demonstrate that histatin has a significant dose dependent accelerated healing activity for corneal wounds.

Ocular surface disorders including, but not limited to, dry eyes, corneal ulcerations and erosions, inflammatory and infectious keratitis and conjunctivitis, surgical interventions (including, but not limited to, laser surgery, glaucoma filtering procedures and tube shunts, cataract surgery, blepharoplasty plastic surgery of the eye lids, Pterigium (Surfer's eye) surgery), and trauma all lead to disruptions in the integrity of the corneal and conjunctival cellular barrier that result in increased risk of infection, pain, and reduced visual acuity. Histatins have a potential use in the treatment of ocular surface trauma/injury and infectious disease.

The outer layer of the cornea, the corneal epithelium, serves as a physical barrier against the environment and thus also as a line of defense to prevent infectious and/or toxic agents from infecting/affecting the tissue. When injury occurs to the surface of the cornea, the corneal epithelium spearheads a wound healing process (see, for example, Klyce S D, Crosson C E. “Transport processes across the rabbit corneal epithelium: a review”. Curr. Eye Res. 1985; 4:323-331 and Lu L, Wang L, Shell B. “UV-induced signaling pathways associated with corneal epithelial cell apoptosis”. Invest. Ophthalmol. Vis. Sci. 2003; 44:5102-5109, both herein incorporated by reference).

The study disclosed herein evaluates and quantifies the wound healing effects of histatins on the ocular surface of New Zealand White rabbits.

Brief Methodology:

Epithelial defects are created in the right eye (oculus dexter, OD) of 12 New Zealand White rabbits. Due to animal regulations, bilateral wounding is not permitted. After the epithelial defects are made, the rabbits are randomized into treatment groups. Two (2) groups are treated with different cyclized histatin concentrations: 0.1 μg/mL and 10 μg/mL dissolved into an ophthalmic artificial tear preparation and delivered to the rabbits as an eye drop three times daily. Histatins known in the art, including, but not limited to, amino acid SEQ ID NOS: 1 through 33, which include Hst-1 (SEQ ID NO: 4), Hst-2 (SEQ ID NO: 5), Hst-3 (SEQ ID NO: 6), Hst-5 (SEQ ID NO: 30), and sortase cyclized histatin (SEQ ID NO: 33), may be used in these studies or in treatment protocols. One (1) group is treated with an inactive/inert formulation (control). This control group should receive the same vehicle identical to the other two groups but without histatin. An over-the-counter artificial tear preferably serves as the vehicle. The initial study included four (4) animals per group in three (3) groups, for a total of twelve rabbits.

The groups are to be treated with agent (either histatin or an inactive/inert formulation of artificial tears) three times/day (TID) for 7 days. Each rabbit group is preferably given moxifloxicin treatment to prevent infection.

The corneal wounds are then evaluated daily for the corneal wound healing abilities of histatin via fluorescein staining, fluorescent slit lamp biomicrophotography and computerized area determination. Evaluators are masked to the therapeutic treatment given to the rabbits. After healing, two (2) animals from each group are euthanized, and the corneas collected for histological processing (H&E staining with subsequent evaluation by veterinary histopathologist). The decision to perform histopathologic analysis after tissue procurement is made only if there is proven difference in healing between the different treatment groups and the controls. At study termination (study preferably continues for seven days), the remaining animals are euthanized.

Results

The data from a first study using the methodology above is shown in Table 1. The histatin used in this study was cyclized histatin 1. The histatin 1 used was a sortase cyclized histatin with amino acid SEQ ID NO: 33, in which the “C-terminal” T is linked to the “N-terminal” G. Table 2 shows the mm size values (without the standard deviation) as an approximate percentage of the size at 1 hour post wound for each of the three groups. Pathological analysis of the rabbit corneas showed no toxicity.

TABLE 1

| Hours Post | Control | 0.1 μg/mL | 10 μg/mL |
|---|---|---|---|
| Wound | (mm²) | (mm²) | (mm²) |
| 0 | 69.3 ± 17.3 | 112.7 ± 37.8 | 88.4 ± 31.6 |
| 6 | 72.6 ± 15.2 | 96.2 ± 13.6 | 74.0 ± 14.7 |
| 24 | 50.6 ± 16.2 | 77.5 ± 25.0 | 51.4 ± 9.3 |
| 30 | 46.6 ± 25.8 | 48.8 ± 8.0 | 37.7 ± 9.4 |
| 48 | 13.6 ± 20.2 | 2.7 ± 3.1 | 0.00 ± 0.07 |
| 54 | 5.2 ± 7.9 | 0.00 ± 0.00 | 0.00 ± 0.00 |
| 72 | 0.00 ± 0.00 | 0.00 ± 0.00 | 0.00 ± 0.00 |

TABLE 2

| | Control- | 0.1 ug/mL- | 10 ug/mL- |
|---|---|---|---|
| | Percentage | Percentage | Percentage |
| Hours | of 1 hour | of 1 hour | of 1 hour |
| Post | post | post | post |
| Wound | wound size | wound size | wound size |
| 0 | 100% | 100% | 100% |
| 6 | 105% | 85% | 84% |
| 24 | 73% | 69% | 58% |
| 30 | 67% | 43% | 43% |
| 48 | 20% | 2% | 0% |
| 54 | 8% | 0% | 0% |
| 72 | 0% | 0% | 0% |

The results above show that histatin demonstrates a significant dose dependent accelerated healing activity of corneal wounds. While Table 2 does not take into account the standard deviations from Table 1, the percentages clearly indicate that the wounds treated with 0.1 μg/mL or 10 μg/mL histatin healed faster (shrunk more) at each of the time points where data was collected. These results are the first results of their kind done using in vivo animal studies.

Histatins and peptide portions or peptide fragments of histatins may be used to accelerate corneal wound healing or ocular surface disease healing in humans and other animals. In preferred embodiments, histatin 1 (Hst-1), histatin 2 (Hst-2), histatin 5 (Hst-5), peptide fragments of Hst1, Hst2, or Hst5, or any combinations thereof may be used. In other embodiments, histatin 3 (Hst-3) or the D-enantiomer of histatin 2 (D-Hst-2), or peptide fragments thereof, may be used. Any combinations of any of the histatins may be used. In preferred embodiments, histatin concentrations between 0.1 μg/mL and 1000 mg/mL may be used. Peptides with amino acid SEQ ID NOS: 1-33, histatins known in the art, the peptides disclosed in WO 2009/087117 or the peptides disclosed in Dr. Menno Johannes Oudhoff's thesis, “Discovery of the Wound-Healing Capacity of Salivary Histatins”, 2010, department of Oral Biochemistry of the Academic Centre for Dentistry Amsterdam (ACTA), VU University Amsterdam and University of Amsterdam, The Netherlands, herein incorporated by reference, may be used.

In one preferred embodiment, histatin 1 (Hst-1) or histatin 2 (Hst-2) in combination with histatin 5 (Hst-5), peptide fragments of Hst-1 or Hst-2 in combination with peptide fragments of Hst-5, or any combination, are used. Hst-5 inhibits production of Matrix Metalloproteases (MMPs).

The combination of the Hst-1/Hst-2 healing properties with the Hst-5 inhibiting MMPs should be very effective. In some preferred embodiments, a concentration of at least approximately 1 μM of Hst-5, or a fragment of Hst-5, is used.

In one preferred embodiment, a cyclic version of SEQ ID NO: 33 is used in combination with SEQ ID NO: 30, either in a cyclized or non-cyclized form.

Histatins could be administered to humans or other animals with a corneal wound, ocular viruses, ocular fungi, ocular parasites, or ocular surface disorders. Some methods of administration include, but are not limited to, incorporating the histatin into eye drops, gels or ointments, incorporating the histatin into tissue glue used to transiently seal corneal injuries, or embedding the histatin into (polymer) contact lenses.

The histatins may be administered in any combination of daily treatments for any number of days in order to produce therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

### In Vivo Uses

In some embodiments, histatin formulations may be used in the treatment of ocular surface trauma/injury and infectious disease.

Photorefractive keratectomy (PRK) is a method of excimer laser corneal refractive surgery, shown to be effective and safe in the correction of refractive errors (see, for example, Rajan M S, Jaycock P, O'Brart D, Nystrom H H, Marshall J. “A long-term study of photorefractive keratectomy; 12-year follow-up”. Ophthalmology. 2004; 111(10):1813-24, herein incorporated by reference). This method has a shorter operation duration, no micro-keratome usage and no flap complications in comparison to laser in situ Keratomileusis. However, short term pain and discomfort, and delayed recovery are some reported disadvantages (see, for example, Krueger R R. “Introduction to commercially approved wavefront-guided customization: third year in review”. J. Refract. Surg. 2005; 21(6):S767-S768, herein incorporated by reference).

Every corneal abrasion, with no damage to the Bowman's layer or stroma, heals in a symmetric fashion, indicating that the rate of cellular migration is equivalent at all points along the wound border (see, for example, Crosson C E, Klyce S D, Beuerman R W. “Epithelial wound closure in the rabbit cornea; a biphasic process”. Invest. Ophthalmol. Vis. Sci. 1986; 27:464-473; and Estil S, Kravik K, Haaskjold E, et al. “Pilot study on the time course of apoptosis in the regenerating corneal epithelium”. Acta Ophthalmol. Scand. 2002; 80:517-523, both herein incorporated by reference). In the renewal process, the epithelial cells are immediately able to establish appropriate connections with adjacent cells, maintaining the epithelial integrity and reducing the corneal surface's impermeability to external agents (see, for example, Lu L, Reinach P S, Kao W W-Y. “Corneal epithelial wound healing”. Exp. Biol. Med. 2001; 226:653-664, herein incorporated by reference). Furthermore, rapid and noncomplicated epithelial healing has been correlated to the reduction in epithelial, stromal remodeling processes (see, for example, Møller-Pedersen T, Vogel M, Li H F, et al. “Quantification of stromal thinning, epithelial thickness, and corneal haze after photorefractive keratectomy using in vivo confocal microscopy”. Ophthalmology. 1997; 104:360-368, herein incorporated by reference) with less haze and refractive regression, allowing prediction of the refractive outcome and its stability more accurately (see, for example, Serrao S, Lombardo M. “One-year results of photorefractive keratectomy with and without surface smoothing using the Technolas 217C laser”. J. Refract. Surg. 2004; 20:444-449; and Detorakis E T, Siganos D S, Kozobolis V P, Pallikaris I G. “Corneal epithelial wound healing after excimer laser photorefractive and photoastigmatic keratectomy (PRK and PARK)”. Cornea. 1999; 18:25-28, both herein incorporated by reference).

Pharmacological agents have been used to control the remaining variations in the wound-healing response. Corticosteroids had been used extensively as a postoperative treatment, but their use has diminished as the surgical technique has changed from steep ablations with a small diameter to flat large-diameter treatments. Wound healing of all body parts follows a similar pattern with local variations. Wound healing in the skin provides the standard description of the discernible phases: (1) inflammation—early, polymorphonuclear leukocyte invasion; late, monocyte invasion; (2) granular tissue formation and re-epithelialization; (3) new matrix formation and remodeling of the matrix; (4) wound contraction; and (5) collagen accumulation and normalization of the number of cells in the scar. All these wound-healing phases occur in the cornea, but not to the same extent. One reason is the lack of vessels in the cornea.

The epithelial covering of the ablated corneal surface area is an early and important step in wound healing. The surgical insult to the cornea stimulates an inflammatory response and adversely affects corneal nerves that are essential to sensing dryness and promoting reflexive tearing. Opacification of the anterior stroma, better known as corneal haze, is a potential postsurgical complication that causes reduced contrast sensitivity, glare, and, in severe cases, reduced visual acuity. The end result can be a neurotrophic cornea, production of haze, and a less predictable refractive outcome.

When epithelial cell coverage is complete, the barrier against infection is restored; irritation, tearing, and photophobia stop; injection subsides; and vision returns. This process usually takes 72-96 hours. However, epithelial healing is not really complete until permanent anchoring is restored, which requires about 6 weeks. The epithelial healing can be divided into a latent phase, lasting about 8 hours, followed by a linear healing phase of migration and proliferation, and finally the establishment of permanent cell attachments (adhesion).

PRK creates or worsens corneal dryness and induces localized inflammation due to disruption of corneal nerves and trauma to the corneal tissue. Appropriately managing both the inflammation and dryness after surgery is critical to achieving excellent visual results. In some embodiments, histatin formulations are used in the management of inflammation and dryness. The outcome of photorefractive keratectomy (PRK) is closely related to the wound-healing response.

In some embodiments, in addition to the standard antibiotic and steroid postoperative therapeutic regimens, an eye of a human patient subject to PRK receives supplemental treatment with histatin in an artificial tear vehicle.

In some preferred embodiments, the formulations in the artificial tear vehicle or other eye drop include a combination of full length Histatin 1 and/or Histatin 2 with Histatin 5. In other embodiments, the formulations include peptide fragments of one or more of Histatin 1 and/or Histatin 2, with Histatin 5. In still other embodiments, both peptide fragments of one or more of Histatin 1, Histatin 2 or Histatin 5 and one or more of full length Histatin 1, Histatin 2 and/or Histatin 5 are used. The peptides or peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33. In one preferred embodiment, the formulation includes SEQ ID NO: 30 and SEQ ID NO: 33 (cyclized).

In some embodiments, a preoperative assessment is performed that includes a complete questionnaire about dryness of the eyes, vision fluctuations, and foreign body sensation and a preoperative ophthalmic examination. The preoperative ophthalmic examination preferably includes manifest and cycloplegic refractions, corneal keratometry and topography, corneal pachymetry, pupillometry, slitlamp biomicroscopy, fundus examination, tonometry, and wavefront analysis. Baseline measurements preferably include uncorrected distance visual acuity (UC-DVA), best-corrected distance visual acuity (BC-DVA), manifest refraction, and corneal topography.

In some embodiments, the patient is asked one or more of the following questions prior to a PRK/histatin procedure: How often do you have problems with dry eyes? If you have problems with dry eyes, how severe is it? How often do you have problems with your vision fluctuating over the day? How often do you have problems with gritty, scratchy, or sandy feelings in your eyes?

In some embodiments, the eyes of the human patient must meet at least one or all the following criteria to qualify for the PRK/histatin procedure:
a stable refraction documented by a change in sphere and cylinder <0.50 D in the previous 12 months
−1D to −8D of spherical myopia
<3.0D of regular astigmatism
a difference in refraction between the eyes of ≦2 diopters
a corneal thickness of both eyes greater or equal to 480 μm
a visual acuity correctable to 20/20 in both eyes by Snellen acuity
discontinuation of any soft contact lens wear ≧3 days before the preoperative evaluation
discontinuation of rigid gas permeable (RGP) or hard contact lenses ≧3 weeks prior to the baseline visit.

In some embodiments, one or more of the following conditions exclude the eyes of the human patient from the PRK/histatin procedure:
any corneal pathology, including scars, prior herpes keratitis, corneal transplants, keratoconus, or corneal dystrophies
a history of refractive or other ocular surgery in either eye
a history of serious eye disease or trauma
any active ocular disease, corneal abnormalities, lid abnormalities, or any ocular pathologies
anticipated wearing of contact lenses post-PRK
wound healing abnormalities, such as keloids
a history of isotretinoin use
an uncontrolled systemic disease, such as Sjogren's disease or rheumatotoid arthritis
any immunocompromised state including diabetes mellitus, cancer, HIV infection, or Hepatitis B or C
a history of unstable myopia
pregnancy or lactation within the previous 6 months
a known allergy or hypersensitivity to the study medication.

Procedure

In some embodiments, the laser surgery procedure includes one or more of the following steps. The lid margins, cul-de-sac, and periocular region are disinfected using diluted povidone. Following a 5% a povidone-iodine scrub of eyelid and skin, topical anesthetic drops are instilled in the upper and lower fornices. A sterile drape is placed to isolate the surgical field. A lid speculum is inserted to allow maximum exposure of the globe.

Topical Tetracaine 1% anesthetic drops are instilled in the upper and lower fornices before surgery. A 9.0 mm laser-assisted subepithelial keratectomy corneal cone is placed centrally on the cornea and filled with 20% diluted alcohol. Downward pressure is applied to create an epithelial indentation. After 30 seconds of exposure, the alcohol is absorbed with a small sponge, the alcohol cone is removed, and the epithelium is debrided using a blunt spatula or wexcel. The cornea is thoroughly rinsed with a balanced salt solution. The area of the epithelial incision is dried with a small sponge. The patient undergoes laser ablation. Mitomycin—C 0.02% is applied to the stromal bed for up to 30 seconds, and then the surface is dried with a sponge and irrigated with a balanced salt solution. A single drop of topical steroid and fluoroquinolone antibiotic is applied to the eye. No bandage contact lens is placed.

In some embodiments, the patient is dispensed a topical steroid and antibacterial treatment. The patient is instructed to self-administer a topical fluoroquinolone dispensed 4 times daily for 1 week and prednisolone 1% four times a day for 1 month, taken 5 minutes apart.

The patient is also instructed to self-administer an artificial tear vehicle or eye drops containing histatin. In one preferred embodiment, the artificial tear vehicle contains 100 μg/mL histatin and is applied four times daily. The artificial tear vehicle is also preferably taken as the third medication 5 minutes after the steroid drop. The first drop of artificial tear vehicle is instilled prior to leaving the surgical facility. The artificial tear vehicle is preferably applied 4 times daily until the epithelial wound heals and then is continued for 90 days twice daily.

In some preferred embodiments, the artificial tear vehicle containing histatin includes a combination of full length Histatin 1 and/or Histatin 2 with Histatin 5. In other embodiments, the artificial tear vehicles include peptide fragments of one or more of Histatin 1 and/or Histatin 2, with Histatin 5. In still other embodiments, both peptide fragments of one or more of Histatin 1, Histatin 2 or Histatin 5 and one or more of full length Histatin 1, Histatin 2 and/or Histatin 5 are used. The peptides or peptide fragments of the histatins in the artificial tear vehicle preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33. In one preferred embodiment, the artificial tear vehicle includes SEQ ID NO: 30 and SEQ ID NO: 33 (cyclized).

In some embodiments, postoperative examinations are performed every 24 hours until re-epithelialization occurs, then again on day 7, and about day 30 and about day 90. In some embodiments, on each postoperative visit, the patient receives fluorescein dye staining and both vertical and horizontal measurements of the wound area are documented. A digital photo of the fluorescein stain corneas is taken. Each photograph is taken twice to ensure the accuracy of the measures. Digital analysis of the images is also performed.

In some embodiments, at each postoperative examination visit, uncorrected visual acuity (UC-VA), best-corrected visual acuity (BC-VA), autorefraction, manifest refraction, and slitlamp biomicroscopy are checked. Corneal topography is repeated at 3 months. In addition, at each postoperative visit patients are surveyed for dry eye and visual fluctuation, using the same preoperative questionnaire and pain intensity measured by patients using a 5-point scale (0=no pain and 4=intolerable pain). Reepithelialization of the cornea is graded from 0 (no epithelial defect) to 4 (complete epithelial defect) and the presence of corneal haze is scored from 0 (totally clear), 0.5 (trace of opacity), and 1 (mild) to 4 (completely opaque cornea) (see, for example, Maldonado M J, Arnau V, Navea A, Martinez-Costa R, Mico F M, Cisneros A L, et al. “Direct objective quantification of corneal haze after excimer laser photorefractive keratectomy for high myopia”. Ophthalmology. 1996; 103:1970-1978, herein incorporated by reference).

Although the above-described procedure is specific for PRK, histatin formulations may analogously be used more generally in treating any laser surgery ocular wounds. In these embodiments, the post-operative laser surgery patient is instructed to self-administer an artificial tear vehicle or eye drops containing histatin. In one preferred embodiment, the artificial tear vehicle contains 100 μg/mL histatin and is applied four times daily. The artificial tear vehicle is also preferably taken as the third medication 5 minutes after the steroid drop. The first drop of artificial tear vehicle is preferably instilled prior to leaving the surgical facility. The artificial tear vehicle is preferably applied 4 times daily until the epithelial wound heals and then is continued for 90 days twice daily.

In some preferred embodiments, the artificial tear vehicle containing histatin includes a combination of full length Histatin 1 and/or Histatin 2 with Histatin 5. In other embodiments, the artificial tear vehicles include peptide fragments of one or more of Histatin 1 and/or Histatin 2, with Histatin 5. In still other embodiments, both peptide fragments of one or more of Histatin 1, Histatin 2 or Histatin 5 and one or more of full length Histatin 1, Histatin 2 and/or Histatin 5 are used. The peptides or peptide fragments of the histatins in the artificial tear vehicle preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33. In one preferred embodiment, the artificial tear vehicle includes SEQ ID NO: 30 and SEQ ID NO: 33 (cyclized).

Although laser surgery is specifically described in this example, the histatins described herein could alternatively be used after other surgical interventions including, but not limited to, glaucoma filtering procedures and tube shunts, cataract surgery, blepharoplasty plastic surgery of the eye lids, and Pterigium (Surfer's eye) surgery,

### Testing and Treatment for Dry Eyes Prior to Surgery

In some embodiments, an ocular patient is tested for dry eye disease prior to ocular surgery. In some embodiments, the ocular surgery is ocular laser surgery. In other embodiments, the ocular surgery is a non-laser ocular surgery. In some embodiments, the ocular surgery is blepharoplasty, cataract surgery, retina attachment cryoscopy, or canaloplasty for glaucoma. Although any test for dry eye disease could be used, in some embodiments, the test for dry eyes is a point of care lateral flow assay. In some embodiments, the assay detects MMP-9, an inflammatory marker that is consistently elevated in the tears of patients with dry eye disease. In some embodiments, the lateral flow assay is of the type marketed under the InflammaDry® trademark (Rapid Pathogen Screening, Inc.).

In some embodiments, one or more steps of the following procedure are followed to run a dry eye test that detects elevated levels of MMP-9 (for example, the InflammaDry® lateral flow assay): A tear sample is collected from the patient's palpebral conjunctiva by gently dabbing a sample collector in multiple locations along the palpebral conjunctiva. The eyelid is released after every 2 to 3 dabs to allow the patient to blink. This is repeated 6 to 8 times, and then the sampling fleece is allowed to rest against the conjunctiva for at least 5 seconds or until the sampling fleece is saturated with tears (5-10 mL). Adequate saturation of the sampling fleece is indicated by a pink color or glistening appearance. Next, the test is assembled by snapping the sample collector onto the provided test cassette. The assembled test is then dipped into the provided test buffer solution for 20 seconds for activation. Last, after 10 minutes have elapsed, the test values are read. The presence of 1 blue line and 1 red line in the test result window indicates a positive test result (matrix metalloproteinase-9 (MMP-9)≧40 ng/mL). The intensity of the red line is directly related to the amount of MMP-9 present, thus, mild dry eye is associated with fainter lines than more severe dry eye is. The presence of a red line of any intensity confirms the presence of elevated MMP-9. One blue line indicates a negative test result (MMP-9<40 ng/mL). The dry eye test is analyzed within 24 hours of activation. The dry eye test preferably has built-in procedural controls, including a blue control line. In the unlikely event that the test is not run properly or the reagents do not work, the blue control line does not appear, indicating an invalid test result.

In some embodiments, if the dry eye test result is positive, the patient is treated with an anti-inflammatory treatment prior to the laser or other ocular surgery. In some embodiments, the anti-inflammatory treatment includes histatin. In some preferred embodiments, the anti-inflammatory treatment include a combination of full length Histatin 1 and/or Histatin 2 with Histatin 5. In other embodiments, the formulations include peptide fragments of one or more of Histatin 1 and/or Histatin 2, with Histatin 5. In still other embodiments, both peptide fragments of one or more of Histatin 1, Histatin 2 or Histatin 5 and one or more of full length Histatin 1, Histatin 2 and/or Histatin 5 are used. The peptides or peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33. In one preferred embodiment, the anti-inflammatory includes SEQ ID NO: 30 and SEQ ID NO: 33 (cyclized).

In other embodiments, the anti-inflammatory treatment includes one or more of the following anti-inflammatories: cyclosporin A, for example a 0.05% cyclosporine ophthalmic emulsion marketed under the trademark Restasis® (Allergan, Inc.), an omega-3 fatty acid, an anti-inflammatory steroid, a corticosteroid, loteprednol, a loteprednol etabonate ophthalmic suspension, for example the suspension marketed under the trademark Lotemax® (Baush & Lomb Inc.), or doxycycline. In other embodiments, the anti-inflammatory treatment may include a combination of any one or more of the above-listed anti-inflammatories with any one or more of the above-listed histatin peptides or peptide fragments.

In some embodiments, if the patient complains of dry eye but the test is negative, a punctal plug or a punctal occlusion is used prior to or during surgery.

### Ocular Histatin Formulations

In some embodiments, a formulation for ocular histatin includes a total of approximately 25-150 μg/mL of a combination of a histatin 1 fragment and a histatin 5 fragment in an applying vehicle. In some preferred embodiments, a formulation for ocular histatin includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation. In some preferred embodiments of this formulation, both the histatin 1 and the histatin 5 fragment are cyclized. In other preferred embodiments, one of the fragments is cyclized. In other embodiments, neither of the fragments is cyclized.

In one preferred embodiment, the histatin 1 fragment is a cyclized version of SEQ ID NO: 33. In another preferred embodiment, the histatin 5 fragment is SEQ ID NO: 30. In another preferred embodiment, the histatin 5 fragment is a cyclized version of SEQ ID NO: 30. In another preferred embodiment, the histatin 1 fragment is a cyclized version of SEQ ID NO: 33 and the histatin 5 fragment is SEQ ID NO: 30 (either in a cyclic or linear form).

The preferred vehicle in ocular histatin formulations is 1.0% carboxymethylcellulose sodium. Other alternative or additional vehicles that may be used for stability include, but are not limited to 0.2% hydroxypropyl methylcellulose, 0.2% glycerin, or 1% polyethylene glycol 400.

Some preferred preservatives to be used in the formulation include, but are not limited to, sodium perborate (for example, GenAqua™ sodium perborate from Novartis, AG or Purite® sodium perborate from Allergan, Inc.), polyquaternium-1 (for example, Polyquad® preservative from Alcon Research, Ltd.), also known as polidronium chloride, or a borate/sorbitol/propylene glycol/zinc preservative (for example, SofZia® preservative from Novartis AG Corp.). Benzalkonium chloride (BAK) is preferably not used in these ocular formulations because of its potentially adverse side effects, including, but not limited to, an observed significant cytotoxicity to cultured ocular epithelial cells in vitro.

Metal ions and metal suspensions such as silver and colloidal gold have been used in sprays, emulsions etc. for their antimicrobial activity. There are therapeutic silver ointments and gels that are used to cleanse and treat wounds. Zinc lozenges (for example, Zicam® zinc lozenges) are used to lessen the duration of flu. In some embodiments, trace amounts of metal ions (generally 1000 to 10,000 times less than the lowest toxic doses) may be used in mucosal type applications including eye drops. These metallic elements are introduced as bound moieties to various proteins or polypeptides. For example, some of the metallo matrix proteases may become inactive in the presence of selenium or manganese. In some embodiments, a metal ion or a metal suspension including, but not limited to, silver, colloidal gold, selenium, manganese, or zinc, may be included in the histatin ocular formulations.

In some embodiments, the ocular histatin formulation is stable both at room temperature (25° C.) and at 45° C. for at least 90 days. One such formulation that has been tested and has been found to be stable, as determined by mass spectroscopy, for at least 90 days at 45° C. and for at least 505 days at 25° C. includes cyclized histatin 1 (SEQ ID NO: 33) at a concentration of 100 μg/mL in a commercially-available gel tears lubricating eye drop with 0.25% polyethylene glycol (PEG) in an aqueous buffered borate salt solution.

These formulations may be administered to humans or other animals with a corneal wound or ocular surface disorders. Some methods of administration include, but are not limited to, incorporating the histatin into eye drops, gels or ointments, incorporating the histatin into tissue glue used to transiently seal corneal injuries, or embedding the histatin into (polymer) contact lenses.

These formulations may be administered in any combination of daily treatments for any number of days in order to produce therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

All of the patent and nonpatent references discussed herein are incorporated herein by reference.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A histatin formulation for treating ocular wounds or ocular surface disease comprising:
a) a first peptide comprising a first histatin selected from the group consisting of:
i) histatin 1;
ii) a fragment of histatin 1;
iii) histatin 2;
iv) a fragment of histatin 2;
v) histatin 1 and histatin 2;
vi) histatin 1 and a fragment of histatin 2 vii) histatin 2 and a fragment of histatin 1;
viii) a fragment of histatin 1 and a fragment of histatin 2; and
b) a second peptide comprising a second histatin selected from the group consisting of:
i) histatin 5; and
ii) a fragment of histatin 5;
wherein the formulation comprises approximately 50-100 μg/mL of a combined formulation of the first histatin and the second histatin.

2. The formulation of claim 1, wherein the formulation comprises 50-75 wt % of the first histatin and 25-50 wt % of the second histatin with respect to a total weight of histatins in the formulation.

3. The formulation of claim 1, wherein a weight-to-weight ratio of histatin 1 to histatin 5 is selected from the group consisting of: 1:1, 2:1, 3:1, 4:1, 5:1, 1:2, 1:3, 1:4, 1:5

4. The formulation of claim 1, wherein at least one of a) and b) is cyclized.

5. The formulation of claim 1, wherein the formulation comprises SEQ ID NO: 30 and SEQ ID NO: 33.

6-12. (canceled)

13. A method of performing corneal laser surgery on at least one eye of a patient comprising the steps of:
a) cutting a cornea of the eye of the patient with a surgical laser; and
b) administering a therapeutic amount of at least one medicament comprising at least one peptide to an ocular surface of the eye, wherein the at least one peptide is selected from the group consisting of:
a peptide comprising at least a first histatin;
a peptide comprising at least a fragment of the first histatin;
a first peptide comprising at least the first histatin and a second peptide comprising at least a second histatin;
a first peptide comprising at least the fragment of the first histatin and a second peptide comprising at least a fragment of the second histatin;
a first peptide comprising at least the first histatin and a second peptide comprising at least the fragment of the second histatin; and
a first peptide comprising at least the fragment of the first histatin and a second peptide comprising at least the second histatin.

14. The method of claim 13, wherein the medicament comprises at least two different amino acid sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

15. The method of claim 13, wherein the medicament comprises:
a) the first histatin selected from the group consisting of:
i) histatin 1;
ii) a fragment of histatin 1;
iii) histatin 2;
iv) a fragment of histatin 2;
v) histatin 1 and histatin 2;
vi) histatin 1 and a fragment of histatin 2
vii) histatin 2 and a fragment of histatin 1; and
viii) a fragment of histatin 1 and a fragment of histatin 2; and
b) the second histatin selected from the group consisting of:
i) histatin 5; and
ii) a fragment of histatin 5.

16. The method of claim 13, wherein the second histatin comprises an amino acid sequence selected from the group consisting of SEQ ID NO: 30 and SEQ ID NO: 31.

17. The method of claim 13, wherein the first histatin comprises an amino acid sequence selected from the group consisting of SEQ ID NO: 4; SEQ ID NO: 5; SEQ ID NO: 8; SEQ ID NO: 9; SEQ ID NO: 13; SEQ ID NO: 29; and SEQ ID NO: 33.

18. The method of claim 15, wherein the first histatin is SEQ ID NO: 33 and the second histatin is SEQ ID NO: 30.

19. The method of claim 13, wherein the medicament comprises:
a) the first histatin selected from the group consisting of:
i) histatin 1;
ii) histatin 2; and
iii) a combination of histatin 1 and histatin 2; and
b) the second histatin comprising histatin 5.

20-21. (canceled)

22. The method of claim 13, further comprising the step of performing a dry eye test on the eye of the patient prior to step a).

23. The method of claim 22, wherein the dry eye test is a point of care lateral flow assay test.

24. The method of claim 23, wherein matrix metalloproteinase-9 is an analyte for the point of care lateral flow assay test.

25. The method of claim 22 further comprising the step of administering an anti-inflammatory formulation prior to step a), if the dry eye test gives a positive result.

26. The method of claim 25, wherein the anti-inflammatory formulation comprises an anti-inflammatory agent selected from the group consisting of:
at least a first histatin;
at least a fragment of the first histatin;
at least the first histatin and a second histatin;
at least the fragment of the first histatin and a fragment of the second histatin;
at least the first histatin and at least the fragment of the second histatin; and
at least the second histatin and at least the fragment of the first histatin.

27. The method of claim 25, wherein the anti-inflammatory formulation comprises an anti-inflammatory agent selected from the group consisting of:
cyclosporin A;
a cyclosporine;
an omega-3 fatty acid;
an anti-inflammatory steroid;
a corticosteroid;
loteprednol;
loteprednol etabonate; and
doxycycline.

28. The method of claim 22 further comprising the step of applying a punctal plug or a punctal occlusion to the eye of the patient prior to step a), if the patient reports dry eye symptoms but the dry eye test gives a negative result.

29. A method of identifying whether a patient that is scheduled to undergo ocular surgery has dry eye on at least one eye of the patient, comprising the steps of:
a) performing a dry eye test on at least one eye of the patient; and
b) if the dry eye test gives a positive result, administering a therapeutic amount of an anti-inflammatory formulation comprising at least one peptide comprising at least one histatin to an ocular surface of at least one eye prior to ocular surgery, wherein the peptide is selected from the group consisting of:
a peptide comprising at least a first histatin;
a peptide comprising at least a fragment of the first histatin;
a first peptide comprising at least the first histatin and a second peptide comprising a second histatin;
a first peptide comprising at least the fragment of the first histatin and a second peptide comprising at least a fragment of the second histatin;
a first peptide comprising at least the first histatin and a second peptide comprising at least the fragment of the second histatin; and
a first peptide comprising at least the fragment of the first histatin and a second peptide comprising at least the second histatin.

30. The method of claim 29, wherein the dry eye test is a point of care lateral flow assay test.

31. The method of claim 30, wherein matrix metalloproteinase-9 is an analyte for the point of care lateral flow assay test.

32. The method of claim 29, further comprising, after steps a) and b), performing ocular surgery on at least one eye of the patient.

33-40. (canceled)

41. The method of claim 29 further comprising the step of applying a punctal plug or a punctal occlusion to the eye of the patient if the patient reports dry eye symptoms but the dry eye test gives a negative result.

42-59. (canceled)
